(19) United States
(12) Patent Application Publication
(10) Pub. No.: US 2010/0266674 A1
(43) Pub. Date: Oct. 21, 2010

(54) L-ODDC PRODRUGS FOR CANCER

(75) Inventor: David C.K. Chu, Statham, GA (US)

Correspondence Address: COLEMAN SUDOL SAPONE, P.C., 714 COLORADO AVENUE, BRIDGE PORT, CT, 06605-1601, US

(73) Assignee: UNIVERSITY OF GEORGIA RESEARCH FOUNDATION, INC., Athens, GA (US)

(21) Appl. No.: 12/310,576

(22) PCT Filed: Aug. 30, 2007
(86) PCT No.: PCT/US2007/019016

§ 371 Date: Jun. 21, 2010

Related U.S. Application Data
(60) Provisional application No. 60/842,085, filed on Sep. 1, 2006.

(51) Int. Cl. A61K 9/127; A61K 45/00; A61K 38/20; A61K 38/21; A61K 39/395; A61K 39/07; A61K 38/44; A61K 38/50; A61K 33/36; A61K 33/24; A61K 38/18; A61K 38/12; A61K 38/14; A61K 31/7048; A61K 31/704; A61K 31/7068; A61K 31/7076; A61K 31/661; A61K 31/655; A61K 31/56; A61K 31/496; A61K 31/4965; A61K 31/519; A61K 31/513; A61K 31/282; A61P 35/02; A61P 35/00

(52) U.S. Cl. 424/450; 424/85.1; 424/85.2; 424/85.7; 424/133.1; 424/246.1; 424/94.4; 424/94.6; 424/623; 424/649; 514/7.7; 514/19.9; 514/20.9; 514/27; 514/34; 514/49; 514/48; 514/110; 514/114; 514/151; 514/171; 514/252.11; 514/252.18; 514/255.06; 514/262.1; 514/45; 514/274; 514/492

(57) ABSTRACT

The main drawback in the use of most nucleoside anticancer agents originates from their hydrophilic nature, of which property requires a high and frequent dosage for an intravenous administration. Unlike other nucleoside anti-tumor agents, troxacitabine appears to predominantly enter tumor cells by passive diffusion rather then by using nucleoside transporters, although this may be model dependent. Accordingly, in the present work, a small library of twenty troxacitabine prodrugs has been synthesized using a parallel approach in order to evaluate the relationship between the lipophilicity of the prodrugs and their antitumor activity. Biological evaluation of the prodrugs on two non-small cell lung cancer cell lines (A549 and SW1573) and in pancreatic cell lines clearly showed better antitumor activity than that of troxacitabine, with IC₅₀ values in the nanomolar range.

### RELATED APPLICATIONS

This application claims the benefit of priority of U.S. provisional application no. US60/842,085, entitled “Troxacitabine Prodrugs for Cancer, Especially Non-small Cell Lung Cancer”, filed Sep. 1, 2006, the entire contents of which are incorporated by reference here.

### FIELD OF THE INVENTION

The present invention relates nucleoside compounds which are useful for the treatment of cancers and in particular, non-small cell lung cancers and pancreatic cancers. The present invention relates to the use of certain prodrug forms of L-OddC or troxacitabine, useful in the treatment of cancers, in particular, non-small cell lung cancer and pancreatic cancer.

### BACKGROUND OF THE INVENTION

Troxacitabine (Troxatyl™; L-OddC; (−)-2′-deoxy-3′-oxacytidine)' is the first L-nucleoside analogue ever shown to have anticancer activity and is currently being evaluated in pivotal Phase II/III clinical trials for the third line treatment of acute myelogenous leukemia (AML) and is in a Phase I/II dose ranging trials in patients with refractory pancreatic cancer. This compound shares the same intracellular activation pathway as common antitumor nucleosides (gemcitabine and cytarabine) leading to the formation of its active triphosphate which is then incorporated into the DNA causing immediate chain termination. ² Unlike the aforementioned nucleosides, troxacitabine has a unique pattern of cellular uptake and metabolism, which may render it not susceptible to the common mechanism of resistance to cytotoxic nucleoside analogues. In fact, it has been shown that troxacitabine can be transported into cells by passive diffusion rather than by nucleoside-specific membrane transporters, such as ENT and CNT, and thus may not be subject to ENT and CNT mediated resistance³. This may however be dependent on the type of cells. Moreover, troxacitabine is resistant to deoxycytidine deaminase (dCD), thus retaining its activity against tumors having high dCD levels.⁴ In contrast to the pharmacokinetic behavior of nucleosides analogues with a D-configuration, which are characterized by rapid disappearance from plasma due to dCD-mediated deamination, troxacitabine exhibited a favourable long plasma half-life (82 h) and a systemic clearance comparable to the glomerular filtration rate.⁵ These data indicate that troxacitabine may have activity against refractory tumors. However, while troxacitabine showed relatively longer intracellular retention and low systemic clearance, pharmacokinetic studies indicated that it is slowly accumulated in cancer cells in comparison to other carrier-transported nucleosides.³ Troxacitabine, like most other anticancer nucleosides, is a hydrophillic agent and must be administered intravenously in a frequent dosage schedule, which may result in greater toxicity than the single dose schedule.⁶

In view of these drawbacks, in the present work a library of twenty troxacitabine prodrugs has been synthesized in order to evaluate the relationship between the lipophilicity of the prodrugs and their antitumor activity. Prodrug strategies have been valuable to overcome undesirable pharmaceutical properties of a variety of drugs, thus optimizing their clinical application.⁷ Examples are capecitabine, a prodrug of 5FU⁸; and CP-4055, a 5′-hydroxyl modified lipophilic prodrug of Ara-C, bypasses the ENT and has activity against Ara-C resistant xenografts.⁹ Similarly a gemcitabine analog, CP-4126, modified at the amino group also bypassed the nucleoside transporters (Bergman, 2004). Structural modifications of anticancer and antiviral nucleoside have been conducted on the sugar as well as heterocyclic moiety^{10,11} However, in our current studies, we decided to protect the amino group of cytosine moiety to increase the lipophilicity of troxacitabine rather than the 5′-hydroxyl group to avoid a variety of esterases in plasma.

### BRIEF DESCRIPTION OF THE FIGURES

FIG. 1 shows in Scheme 1, an outline of the general synthetic chemistry, parallel synthesis, as well as a preferred group of compounds which are useful in the present invention.

FIG. 2 shows the activity profile for the most interesting troxacitabine prodrugs on two non-small cell lung cancer cell lines: ^{a}A549, ^{b}SW1573. IC₅₀s are expressed as mean of 3 experiments+SEM.

FIG. 3 shows a correlation between LogP and IC₅₀ for linear chain aliphatic prodrugs on both non-small cell lung cancer cell lines A549 and SW1573. LogP was estimated using ChemDraw 8.0 ultra. IC₅₀ₛ are expressed as mean of 3 experiments

FIG. 4 shows several prodrug compounds according to the present invention which were tested against pancreatic cancer cell lines. FIG. 4a shows the synthesis of troxacitabine prodrugs. Reagents: i. (RCO)₂O, MeOH, 55° C., 6h. ^{b}Structure of the aliphatic side chains attached to troxacitabine and 4b shows the lipophilicity of the compounds (LogP), LogP values were estimated using Chemdraw 8.0 ultra.

FIG. 5 shows the sensitivity of the ^{a}BxPC-3 and ^{b}Panc-02 pancreatic cancer cell lines to troxacitabine and the lipophilic analogs H, I, J and K (from FIG. 4).

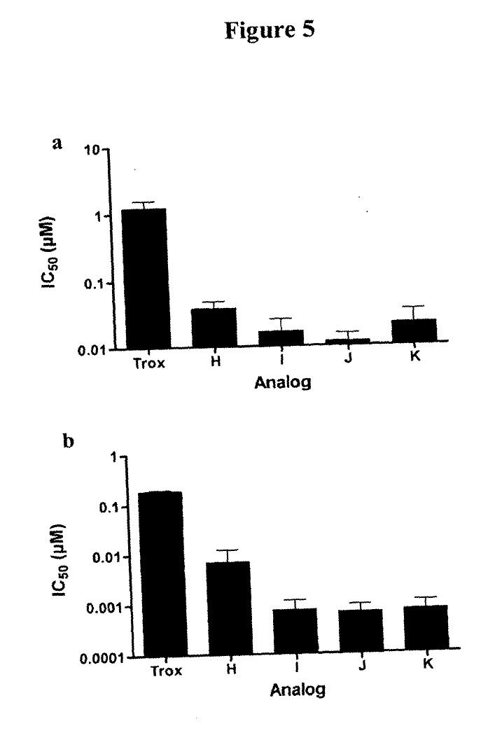

### BRIEF DESCRIPTION OF THE INVENTION

The present invention relates to compounds according to the chemical structure:

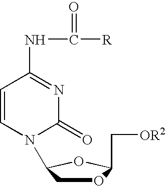

Wherein R is an optionally substituted C₃-C₇ cyclic hydrocarbon, an optionally substituted C₁-C₂₂ straight or branch-chained alkyl group, or an optionally substituted phenyl group; R² is H or a mono-, di- or triphosphosphate in the free base form (as a fully or partially protonated species) or a phosphodiester group, or a pharmaceutically acceptable salt thereof.

Compounds according to the present invention are useful for the treatment of tumors, including cancerous tumors, and especially non-small cell lung cancer or pancreatic cancer.

In a pharmaceutical composition aspect of the invention, pharmaceutical compositions according to the present invention comprise an effective amount of at least one compound as otherwise disclosed herein optionally in combination with a pharmaceutically acceptable carrier, additive excipient.

Methods of treating tumors, including cancer comprise administering to a patient in need of therapy an effective amount of a compound according to the present invention. Cancers which can be treated effectively include a number of cancers, and in particular, non-small cell lung cancer and pancreatic cancer inasmuch as the present prodrug compounds of L-OddC have exhibited unexpected bioavailability in the treatment of these cancers, apparently due to their selective enhanced uptake by cancer cells. This is an unexpected result. Thus, prodrug compounds according to the present invention exhibit enhanced bioavailability consistent with selective uptake especially in non-small cell lung cancer cells and pancreatic cancer cells.

While the compounds according to the present invention may be used to treat numerous cancers, they find particular and exceptional activity because of their enhanced bioavailability (believed to be due to the selective update of these compounds by cancer cells) in the treatment of non-small cell lung cancer and pancreatic cancer, alone or in combination with other anticancer agents.

It is noted that the use of prodrug forms of LOddC as otherwise described herein, when coadministered with another anti-cancer agent in the treatment of cancer in a subject, is substantially more active than the other anti-cancer agent alone, which is an unexpected result. Moreover, a combination of an effective amount of one of the prodrug nucleoside compounds according to the present invention with another anticancer agent (“the other anticancer agent”), in many instances, will provide a synergistic enhancement (i.e., more than additive) of the anticancer activity of the other anticancer agent.

These and/or other aspects of the present invention may be readily gleaned from a review of the detailed description of the invention which follows.

### DETAILED DESCRIPTION OF THE INVENTION

The term “patient” or “subject” is used throughout the specification to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “compound”, as used herein, unless otherwise indicated, refers to any specific chemical compound disclosed herein. Within its use in context, the term generally refers to a single compound preferably, a prodrug of (the L-(3 anomer) the nucleoside L-OddC or its various racemic or enantiomerically enriched (to at least 75%, 85%, 95%, 98%, 99%, 99+%, 100% enantiomeric enrichment) of prodrug forms of L-OddC as otherwise described herein. Compounds according to the present invention exhibit little, if any toxicity, to host cells in treating cancer, an unexpected result and are particularly active against non-small cell lung cancer and pancreatic cancer.

The term “effective” is used herein, unless otherwise indicated, to describe an amount of a compound which, in context, is used to produce or effect an intended result, whether that result relates to the treatment of a tumor including a carcinogenic tumor or other cancer, especially including non-small cell lung cancer or pancreatic cancer. In certain aspects, the present invention relates to combination therapy with another anti-cancer agent or compound. This term subsumes all other effective amount or effective concentration terms which are otherwise described in the present application. With respect to an anti-cancer effect, that effect may be one or more of inhibiting further growth of tumor or cancer cells, reducing the likelihood or eliminating metastasis or producing cell death in the tumor or cancer cells, resulting in a shrinkage of the tumor or a reduction in the number of cancer cells or preventing the regrowth of a tumor or cancer after the patient's tumor or cancer is in remission. As indicated, the compounds according to the present invention may exhibit an anti-cancer effect alone and/or may enhance the ability of another anti-cancer agent to exhibit an anti-cancer effect.

The term “pharmaceutically acceptable salt” is used throughout the specification to describe a salt form of one or more of the compositions (and in particularly preferred aspects according to the present invention, phosphate salts) herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts are particularly preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of neoplasia, including cancer, the term “salt” shall mean a pharmaceutically acceptable salt, consistent with the use of the compounds as pharmaceutical agents. In the case of phosphate groups, the phosphate group may occur in the free acid form (i.e., where all groups are protonated) or in pharmaceutical salt form (where one or more of the free acid groups in the phosphate group is converted to its salt form).

The term “pharmaceutically acceptable derivative” or “derivative” is used throughout the specification to describe any pharmaceutically acceptable prodrug form (such as an ester or ether or other prodrug group) which, upon administration to a patient, provides directly or indirectly the present compound or an active metabolite of the present compound.

The term “alkyl” shall mean within its context a C₁-C₂₂, preferably a C₈-C₁₈ linear, branch-chained or cyclic fully saturated hydrocarbon radical, which may be optionally substituted, such as with a phenyl group, for example.

The term “phosphate ester” or “phosphodiester” is used throughout the specification to describe mono-phosphate groups at the 5′ position of the sugar synthon which are diesterified such that the phosphate group is rendered neutral, i.e., has a neutral charge. Phosphate esters for use in the present invention include those represented by the structure:

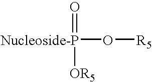

wherein each R₅ is independently selected from H, an optionally substituted C₁ to C₂₀ linear, branched or cyclic alkyl group, an optionally substituted alkoxyalkyl, an optionally substituted aryloxyalkyl, such as phenoxymethyl, an optionally substituted aryl and an optionally substituted alkoxy, among others with the proviso that both R₅ are not H or a pharmaceutically salt thereof. Preferred monophosphate esters (phosphodiesters) for use in prodrug forms according to the present invention are those where R₅ is a C₁ to C₂₀ linear or branched chain alkyl group, more preferably a C₁ to C₃ alkyl group or a pharmaceutically acceptable salt thereof.

The term “optionally substituted” refers to a substituent on an alkyl, alkoxyalkyl, aryloxyalkyl, aryl (esp. phenyl) or alkoxy group which substitutes a moiety other than hydrogen at a chemical position in a compound which otherwise contains a hydrogen. Substituents which may be used in the present invention include, ion context, for example, hydroxyl, carboxyl (C₁-C₆ acid or ester), halogen (F, Cl, Br, I or mixtures thereof), C₁-C₆ (preferably C₁-C₃) alkyl, C₁-C₆ alkoxy or phenyl. It is noted here that each substituent may itself be substituted with a substituent. The term “unsubstituted” refers to the fact that a hydrogen atom is substituted at the indicated position.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embrace's or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, acute lymphocytic leukemia, acute myelogenous leukemia, Ewing's sarcoma, small cell lung cancer, non-small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In particularly preferred aspects of the invention, the target disease is non-small cell lung cancer or pancreatic cancer, two cancers for which the present compounds exhibit exceptional activity.

The term “tumor” is used to describe a malignant or benign growth or tumefacent.

The term “non-small cell lung cancer” is used to describe a disease in which malignant (cancer) cells form in the tissues of the lung. There are several types of non-small cell lung cancer. Each type of non-small cell lung cancer has different kinds of cancer cells. The cancer cells of each type grow and spread in different ways. The types of non-small cell lung cancer are named for the kinds of cells found in the cancer and how the cells look under a microscope:
Squamous cell carcinoma: Cancer that begins in squamous cells, which are thin, flat cells that look like fish scales. This is also called epidermoid carcinoma.
Large cell carcinoma: Cancer that may begin in several types of large cells.
Adenocarcinoma: Cancer that begins in the cells that line the alveoli and make substances such as mucus.
   Other less common types of non-small cell lung cancer are: pleomorphic, carcinoid tumor, salivary gland carcinoma, and unclassified carcinoma. The present invention is useful for the treatment of all types of non-small cell lung cancer.

Therapy for non-small cell lung cancer may include radiation therapy, chemotherapy (especially including compounds according to the present invention or compounds according to the present invention in combination with other anticancer agents, palliative therapy, surgery, laser therapy and biological therapy, among others, including combinations of these therapies.

Anticancer agents which can be used to treat non-small cell lung cancer in combination with prodrug forms of LOddC according to the present invention include for example, ixabepilone, bortezomib, alone or in combination with docetaxel, photofrin (porfimer sodium), taxol (paclitaxel), alone or in combination with cisplatin, gemzar (gemcitabine) and tarceva (erlotinib).

The term “pancreatic cancer” is used to describe malignancy of the pancreas. Pancreatic cancer has been called a “silent” disease because early pancreatic cancer usually does not cause symptoms. If the tumor blocks the common bile duct and bile cannot pass into the digestive system, the skin and whites of the eyes of the patient may become yellow (jaundiced), and the urine darker as a result of accumulated bile pigment called bilirubin.

The pancreas is divided functionally into the endocrine pancreas (that makes insulin and other hormones) and the exocrine pancreas (that makes pancreatic enzymes to aid the digestion). Although the present invention may be useful for treating cancer of the endocrine pancreas, the present invention is primarily useful for the treatment of exocrine pancreas, which is far and away the most common type of pancreatic cancer.

Cancer of the pancreas has markedly increased in incidence over the decades and now ranks as the fourth leading cause of cancer death in the US. Despite the high mortality rate associated with pancreatic cancer, its causation is poorly understood. Smoking is known to be a major risk factor. Cigarette smokers develop cancer of the pancreas two to three times more often than do nonsmokers. Quitting smoking reduces the risk of pancreatic cancer. Cancer of the pancreas is presently rarely curable. The overall survival rate is less than 4%. The cure rates are highest (although still usually under 25%) if the tumor is small (less than 2 cm in diameter) and is truly localized to the pancreas but, such cases account for fewer than 20% of all cases of pancreatic cancer. For patients with advanced cancers, the overall survival rate of all stages is less than 1% at 5 years with most patients dying within 1 year. Staging of the tumor is important to the diagnosis and to the identification of patients with disease that cannot be resected (removed by surgery). Staging has been aided by advances in imaging technology, including the spiral computed tomographic (CT) scan, magnetic resonance imaging (MRI) scan, positron emission tomographic (PET) scan, endoscopic ultrasound, and laparoscopic staging.

There are no specific tumor markers for pancreatic cancer. Markers such as serum CA 19-9 have low specificity. Most patients with pancreatic cancer have an elevated CA 19-9 at diagnosis. Following or during definitive therapy, the increase of CA 19-9 levels may identify patients with progressive tumor growth. However, the presence of a normal CA 19-9 does not rule out recurrence of the tumor.

Patients with any stage of pancreatic cancer are candidates for clinical trials because of the poor response to chemotherapy, radiation therapy, and surgery as conventionally used. However, palliation to ease or relieve symptoms may be achieved with conventional treatment. Palliative measures may include surgical or radiologic biliary decompression, relief of gastric outlet obstruction, and pain control. These and other measures may significantly improve the quality of life.

It is essential to address the potentially disabling psychological events associated with the diagnosis and treatment of pancreatic cancer. The impact of this disease can weigh heavily upon the patient and all those close to him or her.

Many chemotherapeutic agents have been attempted to be used to treat pancreatic cancer without success. Tarceva (erlotinib), alone or in combination with gemcitabine, represents a sometimes effective chemotherapeutic agent(s) against pancreatic cancer. Several natural products including apigenin, MGN-3 (from rice bran) and EGCG (from green tree) are reputed to have significant anticancer effects on pancreatic cancer. Palliation agents, including opioid narcotics and other analgesic agents including NSAIDS, designed to ameliorate pain associated with pancreatic cancer, represent the single best approach to therapeutic intervention in pancreatic cancer. Any one or more of these or other anticancer agents effective for the treatment of pancreatic cancer may be combined with compounds according to the present invention to effect beneficial treatment of pancreatic cancer.

The term “additional anti-cancer compound” or “additional anti-cancer agent” is used to describe any compound (including its derivatives) which may be used to treat cancer and combined with prodrug compounds according to the present invention. Additional anti-cancer compounds as described hereinbelow may be co-administered with one or more of the compounds according to the present invention for the effect that each of these compounds or their derivative compounds have on enhancing the effect of the compounds in treating cancer in a patient pursuant to the present invention. In many instances the co-administration of these compounds or their derivative and another anti-cancer compound results in a synergistic anti-cancer effect. Exemplary anti-cancer compounds for use in the present invention for co-administration with prodrug forms of LOddC as otherwise described herein include anti-metabolites agents which are broadly characterized as antimetabolites, inhibitors of topoisomerase I and II, alkylating agents and microtubule inhibitors (e.g., taxol). Anti-cancer compounds for use in the present invention include, for example, Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others. In preferred aspects of the invention, an effective amount of a prodrug form of LOddC is combined with ixabepilone, bortezomib, alone or in combination with docetaxel, photofrin (porfimer sodium), taxol (paclitaxel), alone or in combination with cisplatin, gemzar (gemcitabine), tarceva (erlotinib) or mixtures thereof in the treatment of non-small cell lung cancer. In the case of the treatment of pancreatic cancer, compounds according to the present invention may be coadministered with one or agents selected from tarceva (erlotinib), alone or in combination with gemcitabine, apigenin, MGN-3 (from rice bran) and EGCG (from green tree) or mixtures thereof. Palliation agents, including opioid narcotics and other analgesic agents including NSAIDS, may also be combined with compounds according to the present invention in the treatment of pancreatic cancer.

The term “coadministration” or “combination therapy” is used to describe a therapy in which at least two active compounds in effective amounts are used to treat cancer as described herein at the same time. The result may be additive or preferably and in most instances, synergistic. Although the term coadministration preferably includes the administration of two active compounds to the patient at the same time, it is not necessary that the compounds be administered to the patient at the same time, although effective amounts of the individual compounds will be present in the patient at the same time. Compounds according to the present invention are preferably administered with one or more anti-cancer agent or palliation agent as otherwise described herein, in effective amounts.

The present invention includes, where relevant, compositions comprising the pharmaceutically acceptable salts of compounds of the present invention. In certain instances, acids are used to prepare the pharmaceutically acceptable acid addition salts of the aforementioned compounds useful in this invention and include those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among others.

The invention also includes compositions comprising base addition salts (especially of the phosphate derivatives) of the present compounds. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

The compounds of this invention primarily related to nucleoside compounds which are characterized as prodrug forms of β-L nucleosides, but can include other stereoisomers where relevant, including optical isomers of the present compounds, as well as racemic, diastereomeric and other mixtures of such isomers, as well as all solvates and polymorphs of the compounds, where relevant.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers, psoriasis or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.5 milligram to about 750 milligrams, more preferably about 1 milligram to about 600 milligrams, and even more preferably about 10 milligrams to about 500 milligrams of active ingredient.

Compounds/compositions according to the present invention are administered in amounts which are effective for treating a particular condition or disease state. The amount of active compound administered will be dependent upon the condition of the patient, the disease state or condition to be treated and the route of administration. The amount of active to be administered may vary from about 0.001 mg/kg/day to as much as 100 mg/kg/day or more of the patient, about 0.005 mg/kg/day to about 10 mg/kg/day, about 0.01 mg/kg/day to about 1 mg/kg/day or any amount which is considered effective within the context of the active compound's use. The compound may be given at a concentration and for a duration which is effective to treat the disease state or condition in the patient. Although compounds according to the present invention may be administered by virtually any route of administration, oral administration is preferred because of the ease of administration and the enhanced patient compliance which generally occurs with this route of administration.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

### Chemistry

The advent of combinatorial techniques has provided a significant impact on the process of drug discovery^{12,13}. In fact, in the past fifteen years, combinatorial chemistry linked to high-throughput screening techniques, has resulted in the discovery of a variety of biologically active compounds¹⁴. Solution-phase combinatorial approaches have recently become of interest as an alternative to the solid phase method for drug discovery and lead optimization. The key advantage of the solution-phase combinatorial approaches over the solid-phase includes the following:¹⁵ 1) an unlimited number of reactions can be used, therefore, providing maximal structural diversity, 2) an unlimited reaction scale allows to produce sufficient quantities of libraries to be tested in a broad range of assays 3) shorter reaction sequences since there is no need for linker manipulation, attachment to and detachment from the resin, 4) intermediates and final products can be obtained directly for purification and assay, 5) traditional analytical techniques (TLC, HPLC-MS, GC-MS and NMR) can be used for monitoring reactions.

We describe herein the synthesis of a library of troxacitabine prodrugs 6a-t using a straightforward parallel solution-phase approach (Scheme 1). Troxacitabine 5, synthesized starting from L-gulose according to a well-known procedure,¹⁶ was dissolved in anhydrous methanol and treated, in an Argonaut Quest 210 organic synthesizer with twenty different acid anhydrides. Some of these anhydrides (2l-n and 4p-s), which are not commercially available, have been prepared by two different procedures as described in Scheme 1.^{17,18} After 6 h at 55° C., the reaction mixtures were simply filtered, and then purified on a small silica flash column with gradient elution. From which, troxacitabine prodrugs 6a-t were prepared and are fully characterized as described in Experimental Section.

### Result and Discussion

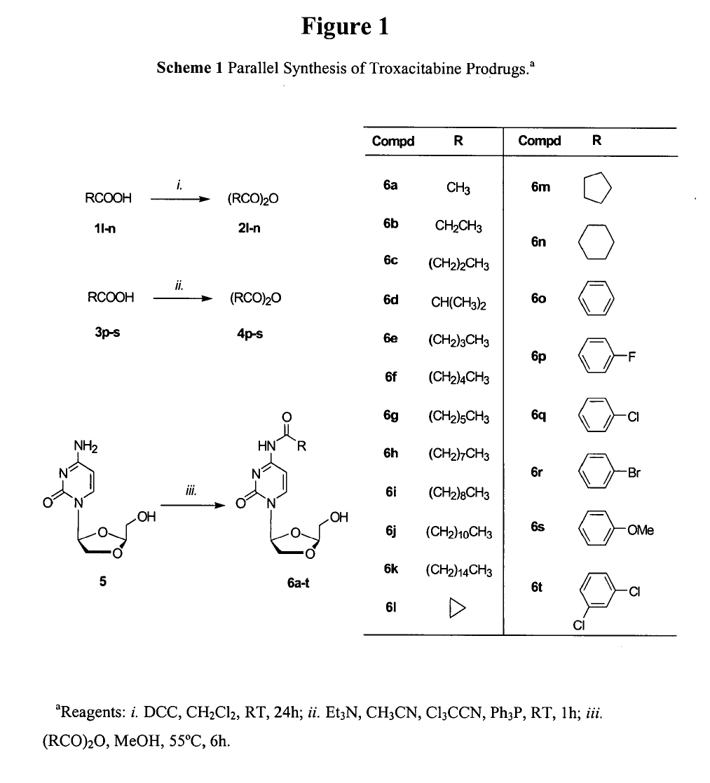

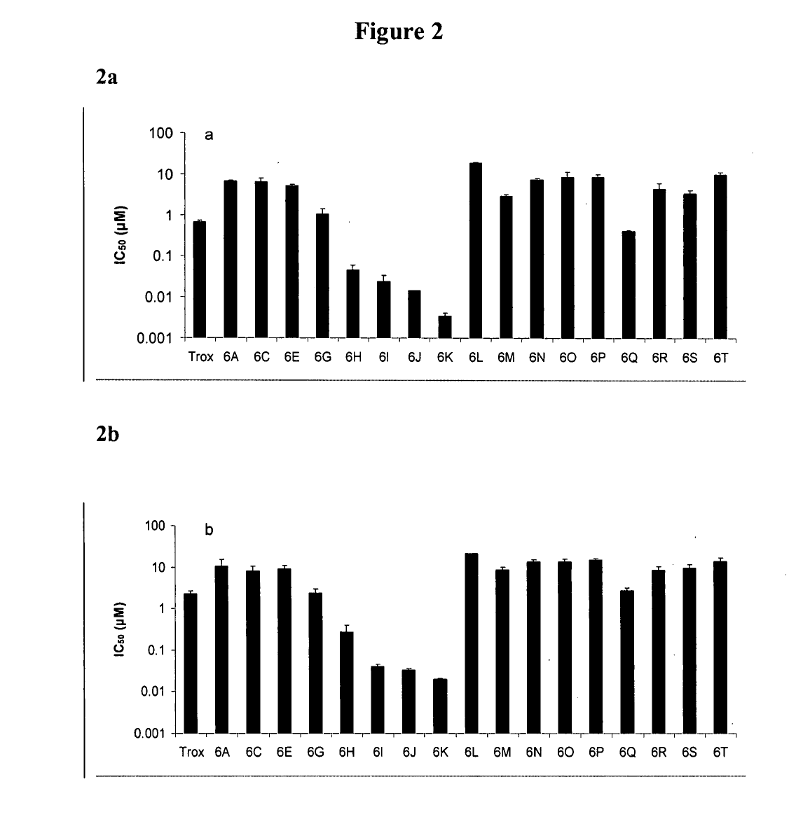

Compounds 6a-t were evaluated using the sulforhodamide-B (SRB) assay in two non-small cell lung cancer cell lines (A549 and SW1573) and the antitumor activity was compared with the parent drug (troxacitabine) and with gemcitabine and cytarabine (Table 1). These cell lines were chosen because they had been characterized earlier for sensitivity to gemcitabine and for the activity of the rate-limiting enzyme in the activation of gemcitabine and troxacitabine, deoxycytidine kinase (dCK); SW1573: 0.3±0.08 nmol/hr/10⁶ cells°, A549 0.99±0.08 nmol/hr/10⁶ cells²⁰. In addition, non-small cell lung cancer is routinely being treated with gemcitabine containing regimens. The growth inhibition (FIG. 1) indicates that analogues 6h-k with long linear aliphatic chains (≧8 CH₂) are clearly more potent than troxacitabine, with IC₅₀ values in the nanomolar range for the best derivatives. It should be noted that it is unlikely that they are being phosphorylated as prodrugs due to their steric hindrance. These preliminary results suggest that lipophilic compounds are taken up more readily than that of troxacitabine. In a cell line we tested whether transport (ENT) would play a role by addition of dipyridamole to the cells. In A549 dipyridamole gave a 2-fold increase in the IC₅₀, but this was not observed for the prodrugs. In fact, a good inverse linear correlation between IC₅₀ and LogP values was found for all the linear-chain aliphatic prodrugs (r²=0.8096 for A549 cell line and r²=0.8199 for SW1573 cell line) as shown in FIG. 2, suggesting that the passive diffusion through the cell membrane would make cells more sensitive to the prodrugs compared to the parent drugs. The compounds that showed the best results in the NSCLC cell lines (6h-k) were also tested in a pancreatic cancer cell line (BxPC3) and were found to have a similar effect. An even greater enhancement in sensitivity than in the NSCLC cell lines was found, compared to troxacitabine a 700-fold decrease in IC₅₀ was observed in compounds 6i-k. It should also be noted that cycloalkyl and aromatic derivatives (6l-t) are less potent than troxacitabine even if some of these compounds possess good lipophilicity. It is likely that these cycloalkyl and aromatic moieties at the N₄ position of compounds 6l-t may render these compounds poor substrates of intracellular amidases. We therefore speculate that the amidase-catalyzed hydrolysis of the prodrugs may play an important role, allowing the release of troxacitabine into the intracellular compartment for activation (the triphosphate). The better activity profile of prodrugs 6h-k could have been the result of an increased uptake due to the high lipophilicity as well as of a high rate of amidase-catalyzed cleavage of the linear aliphatic derivatives intracellularly.

In summary, a straightforward methodology for the parallel synthesis of novel troxacitabine prodrugs 6a-t has been developed. Some of these compounds showed a better antitumor activity against A549 and SW1573 non-small cell lung cancer cell lines. A good inverse linear correlation between LogP and IC₅₀ for linear chain aliphatic prodrugs was also found. In view of these interesting preliminary findings, additional biological evaluations as well as further modifications of troxacitabine are warranted.

### Experimental Section/Examples

### General Considerations

Parallel synthesis was performed on Argonaut Quest 210 organic synthesizer. Melting points were determined on a MeI-temp II laboratory device and are uncorrected. NMR spectra were recorded on a Bruker AMX400 MHz Fourier Transform spectrometer; chemical shifts are reported in parts per million-(8), and signal are quoted as s (singlet), d (doublet), t (triplet), m (multiplet), and dd (double of doublets). UV spectra were obtained on a Beckman DU-650 spectrophotometer. Optical rotations were measured on a JASCO DIP-370 digital polarimeter. TLC was performed on Uniplates (silica gel) purchased from Analtech. Co. and Elemental analysis were performed by Atlantic Microlab Inc., Norcross, Ga. All commercially available anhydrides were used without further purification. Cyclopropanecarboxylic, cyclopenthanecarboxylic and cyclohexanecarboxylic anhydrides were synthesized according to a reported procedure and used without further purification.¹⁶ 4-Fluoro, 4-chloro, 4-bromobenzoic anhydride as well as 2,4-dichlorobenzoic anhydride were synthesized according to a reported procedure.¹⁷

### Experimental Synthetic Methods

General Procedure for the Parallel Solution-Phase Synthesis of 4-(N-Acyl Substituted)-L-OddC Prodrugs (6a-t).
Compound 5 (2 g) was dissolved in anhydrous MeOH (20 mL) and then 1 mL of this solution (100 mg, 1 eq of 5) was added to each microfrit-equipped reaction vessel (RV), followed by 9 mL of methanol. The appropriate anhydrides (3 equivalents) were then added and the reaction mixture was stirred vigorously (upward stroke=50%, time=1 s) at 55° C. for 6 h. After 6 h, the RV's were drained and the collected crude were evaporated to dryness under reduced pressure and then purified on a short flash column (gradient elution, 60% hexane: 40% ethyl acetate-100% ethyl acetate).
(−)-(2S,4S)-1-[2-(Hydroxymethyl)-1,3-dioxolan-4-yl]-4-N-acetyl-cytosine (6a): White solid. Yield=92%; mp 176.0-178.0° C.; [α]²⁴_{D}-68.005 (c 0.04, MeOH); UV (H₂O) λₘₐₓ 245 nm (ε 9033 pH 2), 246 nm (ε 16703 pH 7.4), 270 nm (ε 9760 pH 11); ¹H-NMR(CDCl₃) δ 8.60 (d, 1H, 7.32 Hz), 6.24 (m, 1H), 5.12 (m, 1H), 4.33-4.25 (m, 2H), 3.90 (m, 2H), 2.21 (s, 3H); ¹³C NMR (CD₃OD) δ 180.1, 171.6, 163.1, 156.8, 145.1, 106.1, 96.2, 83.4, 72.0, 60.2, 23.2; IR (neat) 1716, 1654, 1562, 1494 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-propionyl-cytosine (6b): White solid. Yield=56%; mp 161.5-163.5° C.; [α]²⁸_{D}-105.02 (ε 0.022, MeOH); UV (H₂O) λₘₐₓ 246 nm (ε 14465 pH 2), 246 nm (ε 15845 pH 7.4), 270 nm (ε 8368 pH 11); ¹H-NMR (CDCl₃) δ 8.46 (d, 1H, J=7.32 Hz), 7.43 (d, 1H, J=7.32 Hz), 6.20 (dd, 1H, J₁=1.47 Hz, J₂=4.90 Hz), 5.13 (m, 1H), 4.35-4.24 (m, 2H), 3.99 (m, 2H), 2.48 (q, 2H, J=7.32 Hz); 1.20 (t, 3H, J=7.32 Hz); ¹³C NMR (CDCl₃) δ 171.6, 162.6, 156.1, 144.9, 105.8, 96.4, 83.5, 72.7, 60.8, 30.8, 8.8; IR (neat) 1694, 1651, 1556, 1493 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-butirryl-cytosine (6c): White solid. Yield=82%; mp 118.0-120.0° C.; [α]²⁴_{D}-48.43 (c 0.042, MeOH); UV (H₂O) λₘₐₓ 246 nm (ε 16430 pH 2), 246 nm (ε 18353 pH 7.4), 244 nm (ε 10155 pH 11); ¹H-NMR (CDCl₃) δ 8.49 (d, 1H, J=7.33 Hz), 7.44 (d, 1H, J=7.33 Hz), 6.20 (m, 1H), 5.12 (m, 1H), 4.34-4.23 (m, 2H), 3.97 (m, 2H), 2.45 (t, 2H, J=7.32 Hz); 1.70 (m, 2H), 0.97 (t, 3H, J=7.32 Hz); ¹³C NMR (CDCl₃) δ 171.6, 162.5, 155.6, 144.9, 105.7, 96.2, 83.6, 72.7, 61.1, 39.6, 18.3, 13.6; IR (neat) 1693, 1651, 1554, 1500 cm⁻¹. Anal. Calcd for C₁₂H₁₇N₃O₅: C, 50.88; H, 6.05; N, 14.83.

Found: C, 50.90; H, 6.15; N, 13.97.

(−)-(2S,4S)-1-[2-(Hydroxymethyl)-1,3-dioxolan-4-yl]-4-N-isobutirryl-cytosine (6d): White solid. Yield=94%; mp 78.0° C.; (decomposition) [α]²⁷_{D}-55.38 (c 0.042, MeOH); UV (H₂O) λₘₐₓ 246 nm (ε 14907 pH 2), 246 nm (ε 16108 pH 7.4), 244 nm (ε 13387 pH 11); ¹H-NMR (CDCl₃) δ 8.50 (d, 1H, J=7.33 Hz), 7.44 (d, 1H, J=7.33 Hz), 6.21 (dd, 1H, J=1.46 Hz, J₂=5.36 Hz), 5.12 (m, 1H), 4.33 (m, 1H), 4.25 (m, 1H), 3.98 (m, 2H), 2.66 (m, 1H); 1.21 (d, 6H, J=6.83 Hz); ¹³C NMR (CDCl₃) δ 171.6, 162.8, 155.8, 145.3, 105.9, 96.4, 83.5, 72.6, 60.9, 36.5, 19.1; IR (neat) 1739, 1654, 1558, 1490 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-valeryl-cytosine (6e): White solid. Yield=84%; mp 143.0-144.0° C.; [α]²⁴_{D}-53.34 (c 0.5, MeOH); UV (H₂O) λₘₐₓ 24.6 nm (ε 13393 pH 2), 246 nm (ε 14436 pH 7.4), 246 nm (ε 12188 pH 11); ¹H-NMR (CDCl₃) δ 8.49 (d, 1H, J=7.32 Hz), 7.44 (d, 1H, J=7.32 Hz), 6.20 (m, 1H), 5.12 (m, 1H), 4.32 (m, 1H), 4.23 (m, 1H), 3.97 (m, 2H), 2.47 (t, 2H, J=7.32 Hz); 1.65 (m, 2H), 1.36 (m, 2H); ¹³C NMR (CDCl₃) δ 173.8, 162.8, 155.6, 145.2, 105.8, 96.4, 83.5, 72.6, 61.0, 37.4, 26.9, 22.2, 13.7; IR (neat) 1690, 1648, 1552, 1494 cm⁻¹. Anal. Calcd for C₁₂H₁₇N₃O₅: C, 50.88; H, 6.05; N, 14.83.

Found: C, 50.90; H, 6.15; N, 13.97.

(−)-(2S,4S)-1-[2-(Hydroxymethyl)-1,3-dioxolan-4-yl]-4-N-hexanoyl-cytosine (61): White solid. Yield=76%; mp 158.0-160.0° C.; [α]²⁶_{D}-84.09 (c 0.028, MeOH); UV (H₂O) λₘₐₓ 246 nm (ε 14043 pH 2), 246 nm (ε 14495 pH 7.4), 246 nm (ε 12069 pH 11); ¹H-NMR (CDCl₃) δ 8.50 (d, 1H, J=7.32 Hz), 7.45 (d, 1H, J=7.32 Hz), 6.20 (m, 1H), 5.12 (m, 1H), 4.32 (m, 1H), 4.23 (m, 1H), 3.97 (m, 2H), 2.46 (t, 2H, J=7.32 Hz); 1.66 (m, 2H), 1.32 (m, 4H), 0.89 (t, 3H, J=7.32 Hz); ¹³C NMR (CDCl₃) δ 173.8, 162.8, 155.6, 145.3, 105.8, 96.4, 83.5, 72.6, 61.0, 37.6, 31.2, 24.6, 22.4, 13.9; IR (neat) 1693, 1649, 1553, 1494 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-heptanoyl-cytosine (6g): White solid. Yield=84%; mp 151.0-153.0° C.; [α]²⁵_{D}-56.82 (c 0.044, MeOH); UV (H₂O) λₘₐₓ 246 nm (ε 14223 pH 2), 246 nm (ε 15665 pH 7.4), 246 nm (ε 14519 pH 11); ¹H-NMR (CDCl₃) δ 8.49 (d, 1H, J=7.32 Hz), 7.44 (d, 1H, J=7.32 Hz), 6.19 (m, 1H), 5.12 (m, 1H), 4.33 (m, 1H), 4.23 (m, 1H), 3.97 (m, 2H), 2.46 (t, 2H, J=7.32 Hz); 1.66 (m, 8H), 1.28 (m, 4H), 0.89 (t, 311, J=7.32 Hz); ¹³C NMR (CDCl₃) δ 173.7, 162.7, 155.6, 145.2, 105.8, 96.4, 83.5, 72.7, 61.0, 37.7, 31.5, 28.8, 24.8, 22.5, 14.0; IR (neat) 1693, 1649, 1553, 1494 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-nonanoyl-cytosine (6h): White solid. Yield=72%; mp 135.0-137.0° C.; [α]²⁷_{D}-51.58 (c 0.03, MeOH); UV (H₂O) λₘₐₓ 246 nm (ε 14101 pH 2), 246 nm (ε 15201 pH 7.4), 246 nm (ε 11582 pH 11); ¹H-NMR (CDCl₃) 8.49 (d, 1H, J=7.81 Hz), 7.45 (d, 1H, J=7.81 Hz), 6.19 (m, 1H), 5.12 (m, 1H), 4.33 (m, 1H), 4.23 (m, 1H), 3.97 (m, 2H), 2.46 (t, 2H, J=7.32 Hz); 1.66 (m, 2H), 1.28 (m, 10H), 0.88 (t, 3H, J=7.32 Hz); ¹³C NMR (CDCl₃) δ 173.7, 162.7, 155.6, 145.2, 105.8, 96.3, 83.5, 72.7, 61.0, 37.7, 31.8, 29.1, 24.9, 22.6, 14.1; IR (neat) 1689, 1649, 1553, 1496 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-decanoyl-cytosine (61): White solid. Yield=66%; mp 141.0-143.0° C.; [α]²⁵_{D}-35.59 (c 0.028, MeOH); UV (H₂O) λₘₐₓ 246 nm (ε 11850 pH 2), 246 nm (ε 13056 pH 7.4), 246 nm (ε 8256 pH 11); ¹H-NMR (CDCl₃) δ 8.49 (d, 1H, J=7.32 Hz), 7.44 (d, 1H, J=7.32 Hz), 6.19 (m, 1H), 5.12 (m, 1H), 4.33 (m, 1H), 4.24 (m, 1H), 3.97 (m, 2H), 2.45 (t, 2H, J=7.32 Hz); 1.66 (m, 2H), 1.29 (m, 12H), 0.88 (t, 3H, J=7.32 Hz); ¹³C NMR (CDCl₃) δ 173.6, 162.7, 155.6, 145.2, 105.7, 96.3, 83.5, 72.7, 61.0, 37.8, 31.9, 29.4, 29.3, 29.2, 29.1, 24.9, 22.7, 14.1; IR (neat) 1690, 1650, 1553, 1499 cm⁻¹. Anal. Calcd for C₁₂₋₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-lauryl-cytosine (6j): White solid. Yield=54%; mp 137.0-138.5° C.; [α]²⁷_{D}-61.75 (c 0.039, MeOH); UV (MeOH) λₘₐₓ 241 nm (ε 7442); ¹H-NMR (CDCl₃) δ 8.48 (d, 1H, J=7.32 Hz), 7.45 (d, 1H, J=7.32 Hz), 6.19 (m, 1H), 5.12 (m, 1H), 4.33 (m, 1H), 4.24 (m, 110, 3.97 (m, 2H), 2.46 (t, 2H, J=7.32 Hz); 1.66 (m, 2H), 1.25 (m, 16H), 0.88 (t, 3H, J=7.32 Hz); ¹³C NMR (CDCl₃) δ 173.6, 162.7, 155.7, 145.2, 105.8, 96.3, 83.5, 72.7, 61.0, 37.7, 31.9, 29.6, 29.5, 29.4, 29.1, 24.9, 22.7, 14.1; IR (neat) 1690, 1650, 1553, 1499 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-palmitoyl-cytosine (6k): White solid. Yield=23%; mp 134.5-135.5° C.; [α]²⁹_{D}-79.34 (c 0.02, MeOH); UV (MeOH) λₘₐₓ 244 nm (ε 11145); ¹H-NMR (CDCl₃) δ 8.46 (d, 1H, J=7.32 Hz), 7.43 (d, 1H, J=7.32 Hz), 6.19 (m, 1H), 5.13 (m, 1H), 4.34 (m, 1H), 4.26 (m, 1H), 3.98 (m, 2H), 2.42 (t, 2H, J=7.32 Hz); 1.66 (m, 2H), 1.25 (m, 24H), 0.88 (t, 3H, J=7.32 Hz); ¹³C NMR (CDCl₃) δ 162.4, 150.0, 145.0, 105.6, 96.0, 83.6, 72.8, 61.1, 37.9, 31.9, 29.7, 29.6, 29.5, 29.4, 29.3, 29.1, 24.9, 22.7, 14.1; IR (neat) 1690, 1651, 1553, 1499 cm⁻¹. Anal. Calcd for C₁₂H₁₇N₃O₅: C, 50.88; H, 6.05; N, 14.83. Found: C, 50.90; H, 6.15; N, 13.97.
(−)-(2SAS)-1-[2-(Hydroxymethyl)-1,3-dioxolan-4-yl]-4-N-cyclopropyl-cytosine (61): White solid. Yield=66%; mp 169.0-170.5° C.; [α]²⁶_{D}-62.83 (c 0.033, MeOH); UV (H₂O) λₘₐₓ 247 nm (ε 16457 pH 2), 247 nm (ε 18447 pH 7.4), 247 nm (ε 16191 pH 11); ¹H-NMR (CDCl₃) δ 8.48 (d, 1H, J=7.32 Hz), 7.41 (d, 1H, J=7.32 Hz), 6.21 (m, 1H), 5.10 (m, 1H), 4.31 (m, 1H), 4.23 (m, 1H), 3.95 (m, 2H), 1.86 (m, 1H), 1.07 (m, 2H), 0.92 (m, 2H); ¹³C NMR (CDCl₃) δ 174.6, 162.7, 155.8, 145.2, 105.9, 96.7, 83.4, 72.5, 61.0, 15.9, 9.7; IR (neat) 1709, 1651, 1560, 1491 cm⁻¹ Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-cyclopentyl-cytosine (6m): White solid. Yield=58%; mp 59.0° C. (decomposition); [α]²⁷_{D}-31.48 (c 0.031, MeOH); UV (H₂O) λₘₐₓ 247 nm (ε 15014 pH 2), 247 nm (ε 16397 pH 7.4), 247 nm (ε 12296 pH 11); ¹H-NMR (CDCl₃) δ 8.49 (d, 1H, J=7.33 Hz), 7.43 (d, 1H, J=7.33 Hz), 6.20 (m, 1H), 5.12 (m, 1H), 4.31 (m, 1H), 4.24 (m, 1H), 3.97 (m, 1H), 2.86 (m, 1H), 1.92-1.59 (m, 8H); ¹³C NMR (CDCl₃) δ 176.7, 162.7, 155.8, 145.2, 105.8, 96.4, 83.5, 72.6, 61.0, 46.7, 30.1, 26.0; IR (neat) 1717, 1650, 1558, 1489 cm⁻¹. Anal. Calcd for C₁₂H₁₇N₃O₅: C, 50.88; H, 6.05; N, 14.83.

Found: C, 50.90; H, 6.15; N, 13.97.

(−)-(2S,4S)-1-[2-(Hydroxymethyl)-1,3-dioxolan-4-yl]-4-N-cyclohexyl-cytosine (6n): White solid. Yield=64%; mp 77.0° C. (decomposition); [α]²⁷_{D}-76.27 (c 0.039, MeOH); UV (H₂O) λₘₐₓ 247 nm (ε 14885 pH 2), 247 nm (ε 15887 pH 7.4), 247 nm (ε 14220 pH 11); ¹H-NMR (CDCl₃) δ 8.49 (d, 1H, J=7.32 Hz), 7.43 (d, 11-1, J=7.32 Hz), 6.20 (m, 1H), 5.12 (m, 1H), 4.32 (m, 1H), 4.24 (m, 1H), 3.97 (m, 1H), 2.39 (m, 1H), 1.90 (m, 2H), 1.80 (m, 2H), 1.69 (m, 1H), 1.45 (m, 2H), 1.24 (m, 3H); ¹³C NMR (CDCl₃) δ 176.4, 162.8, 155.8, 145.1, 105.8, 96.4, 83.5, 72.6, 61.0, 46.2, 29.2, 25.6, 25.4; IR (neat) 1738, 1654, 1558, 1489 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-benzoyl-cytosine (6o): White solid. Yield=85%; mp 194.5-195.5° C.; [α]²⁸_{D}-56.71 (ε 0.060, MeOH); UV (H₂O) λₘₐₓ 258 nm (ε 19660 pH 2), 258 nm (ε 19802 pH 7.4), 311 nm (ε 14641 pH 11); ¹H-NMR (CD₃OD) δ 8.67 (d, 1H, J=7.32 Hz), 7.98 (d, 2H, J=7.80 Hz), 7.63 (m, 2H), 7.54 (m, 2H), 6.24 (m, 1H), 5.11 (m, 1H), 4.31 (m, 1H), 4.27 (m, 1H), 3.90 (m, 2H); ¹³C NMR (CD₃OD) δ 134.0, 129.7 129.0, 107.3, 98.0, 84.6, 73.3, 61.4; IR (neat) 1699, 1654, 1558, 1488 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-(p-fluorobenzoyl)-cytosine (6p): White solid. Yield=61%; mp 163.0-165.0° C.; [α]²⁷_{D}-96.55 (c 0.036, MeOH); UV (H₂O) λₘₐₓ 259 nm (ε 23324 pH 2), 258 nm (ε 24194 pH 7.4), 314 nm (ε 21234 pH 11); ¹H-NMR (CD₃OD) δ 8.57 (d, 1H, J=7.32 Hz), 7.98 (m, 2H), 7.63 (m, 2H), 7.55 (d, 1H, J=7.32 Hz), 6.14 (m, 1H), 5.04 (m, 1H), 4.24 (m, 1H), 4.18 (m, 1H), 3.87 (m, 2H); ¹³C NMR (CD₃OD) δ 145.4, 130.8, 116.1, 116.0, 106.3, 97.3, 83.6, 72.7, 60.7; IR (neat) 1695, 1650, 1560, 1487 cm⁻¹. Anal. Calcd for C₁₅H₁₄BrN₃O₅: C, 45.47; H, 3.56; N, 10.61. Found: C, 45.57; H, 3.55; N, 10.45.
(−)-(2S,4S)-1-[2-(Hydroxymethyl)-1,3-dioxolan-4-yl]-4-N-(p-chlorobenzoyl)-cytosine (6q): White solid. Yield=29%; mp 190.0-191.5° C.; [α]²⁸_{D}-84.15 (c 0.021, MeOH); UV (H₂O) λₘₐₓ 263 nm (ε 24465 pH 2), 263 nm (ε 24744 pH 7.4), 314 nm (ε 18485 pH 11); ¹H-NMR (CD₃OD) δ 8.53 (d, 1H, J=7.32 Hz), 7.88 (m, 2H), 7.54 (d, 1H, J=7.32 Hz), 7.42 (m, 2H), 6.16 (m, 1H), 5.06 (m, 1H), 4.26 (m, 1H), 4.21 (m, 1H), 3.88 (m, 2H); ¹³C NMR (CD₃OD) δ 132.5, 130.3, 129.7, 107.0, 97.9, 84.3, 73.1, 61.1; IR (neat) 1698, 1651, 1557, 1483 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-(p-bromobenzoyl)-cytosine (6r): Light yellow solid. Yield=56%; mp 189.0-191° C.; [α]²⁶_{D}-74.00 (c 0.039, MeOH); UV (H₂O) λₘₐₓ 265 nm (ε 28850 pH 2), 265 nm (ε 30085 pH 7.4), 316 nm (c 23659 pH 11); ¹H-NMR (CD₃OD) δ 8.66 (d, 1H, J=7.32 Hz), 7.88 (m, 2H), 7.71 (m, 2H), 7.59 (d, 1H, J=7.32 Hz), 6.24 (m, 1H), 5.10 (m, 1H), 4.31 (m, 1H), 4.25 (m, 1H), 3.88 (m, 2H); ¹³C NMR (CD₃OD) δ 146.4, 132.9, 130.7, 107.2, 98.0, 84.5, 73.2, 61.2; IR (neat) 1697, 1651, 1557, 1483 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-(p-methoxybenzoyl)-cytosine (6s): White solid. Yield=68%; mp 181.0-181.5° C.; [α]²⁷_{D}-54.79 (c 0.028, MeOH); UV (H₂O) λₘᵢₙ 303 nm (ε 28704 pH 2), 302 nm (ε 29283 pH 7.4), 302 nm (ε 25191 pH 11); ¹H-NMR (CD₃OD) δ 8.63 (d, 1H, J=7.32 Hz), 7.97 (m, 2H), 7.58 (d, 1H, J=7.32 Hz), 7.05 (m, 2H), 6.24 (m, 1H), 5.10 (m, 1H), 4.31 (m, 1H), 4.25 (m, 1H), 3.89 (m, 5H); ¹³C NMR (CD₃OD) δ 146.5, 131.3, 115.1, 107.5, 98.1, 84.8, 73.3, 61.6, 56.1; IR (neat) 1646, 1567, 1488 cm⁻¹. Anal. Calcd for C₁₂H₁₇N₃O₅: C, 50.88; H, 6.05; N, 14.83. Found: C, 50.90; H, 6.15; N, 13.97.
(−)-(2S,4S)-1-[2-(Hydroxymethyl)-1,3-dioxolan-4-yl]-4-N-(2,4-dichlorobenzoyl)-cytosine (6t): White solid. Yield=84%; mp 185.0-187.0° C.; [α]²⁵_{D}-83.18 (c 0.036, MeOH); UV (H₂O) λₘₐₓ 253 nm (ε 22561 pH 2), 253 nm (ε 23043 pH 7.4), 305 nm (ε 21989 pH 11); ¹H-NMR (CD₃OD) δ 8.71 (d, 1H, J=7.32 Hz), 7.62 (m, 2H), 7.56 (d, 1H, J=7.32 Hz), 7.49 (m, 1H), 6.25 (m, 1H), 5.13 (m, 1H), 4.34 (m, 1H), 4.28 (m, 1H), 3.92 (m, 2H); ¹³C NMR (CD₃OD) δ 147.0, 131.5, 131.0, 128.6, 107.5, 97.8, 84.8, 73.3, 61.5; IR (neat) 1705, 1647, 1557, 1490 cm⁻¹. Anal. Calcd for C₁₅Hₜ₃Cl₂N₃O₃: C, 46.65; H, 3.39; N, 10.88. Found: C, 47.04; H, 3.42; N, 10.82.

### Biological Evaluation—Non-Small Cell Lung Cancer

The compounds were evaluated on two non-small cell lung cancer cell lines (A549 and SW1573). These cell lines were characterized for deoxynucleoside analog sensitivity and activity of dCK previously.

The chemosensitivity assay used in this study was the sulforhodamineB (SRB) assay as described earlier (Keepers et al, Eur J. Cancer, 1991; Rubinstein et al, J. Natl. Cancer Inst., 1990). Cells were transferred to 96 wells plates on day 0; on day 1 a serial dilution of the drug was made from a stock solution and added to the cells in triplicate. After an incubation period of 72 h the cells were fixed for 1 hr at 4° C. with 50% trichloroacetic acid washed, air-dried and stained with 0.4% SRB. The optical density was measured at 492 nm with a microplate reader (Tecan, Salzburg, Austria).

The results were expressed as percentage of control growth:

$$\begin{matrix} & ( {OD ( day 4 )}_{treated} - OD ( day 1 ) ) & \\ \% growth inhibition = & ( {OD ( day 4 )}_{control} - OD ( day 1 ) ) & \times 100 \%\end{matrix}$$

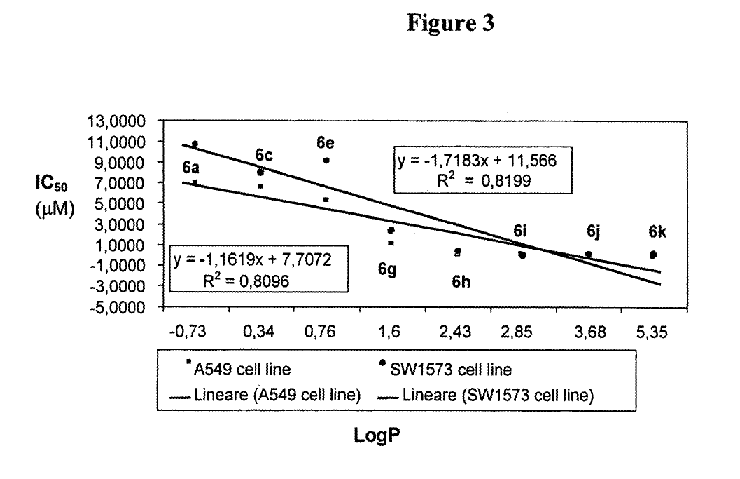

The data were plotted in a graph to give a growth inhibition curve. From this growth inhibition curve the 1050 value was determined by interpolating at the 50% growth level. The results are presented in Table 1, below. FIG. 3 shows a correlation between LogP and IC₅₀ for linear chain aliphatic prodrugs on both non-small cell lung cancer cell lines A549 and SW 1573. LogP was estimated using ChemDraw 8.0 ultra. IC₅₀ₛ are expressed as mean of 3 experiments.

TABLE 1
Cytotoxic activity on two non-small cell lung cancer cell lines (A549
and SW1573) and calculated LogP for Troxacitabine prodrugs 6a-t.

| | IC50(μM),^{a} | IC50(μM),^{a} | |
|---|---|---|---|
| Cpd. | A549 | SW1573 | LogP^{b} |
| Troxacitabine | 0.68 | 2.3 | −0.66 |
| Gemcitabine | 0.013 | 0.008 | −0.90 |
| Cytarabine | 0.62 | 10.50 | −2.24 |
| 6a | 6.87 | 10.70 | −0.73 |
| 6c | 6.60 | 8.07 | 0.34 |
| 6e | 5.23 | 9.13 | 0.76 |
| 6g | 1.04 | 2.40 | 1.60 |
| 6h | 0.046 | 0.276 | 2.43 |
| 6i | 0.024 | 0.040 | 2.85 |
| 6j | 0.015 | 0.033 | 3.68 |
| 6k | 0.004 | 0.020 | 5.35 |
| 6l | 18.67 | 22.00 | 0.00 |
| 6m | 2.97 | 8.80 | 0.83 |
| 6n | 7.37 | 14.00 | 1.25 |
| 6o | 8.53 | 13.67 | 1.17 |
| 6p | 8.67 | 15.33 | 1.33 |
| 6q | 0.41 | 2.77 | 1.73 |
| 6r | 4.53 | 8.87 | 2.00 |
| 6s | 3.50 | 10.03 | 1.04 |
| 6t | 9.83 | 14.33 | 2.29 |

^{a}Evaluated from SRB test and expressed as mean of 3 experiments.
^{b}Calculated using Chem Draw Ultra 8.0.

### Experimental Section Pancreatic Cancer

### Materials and Methods

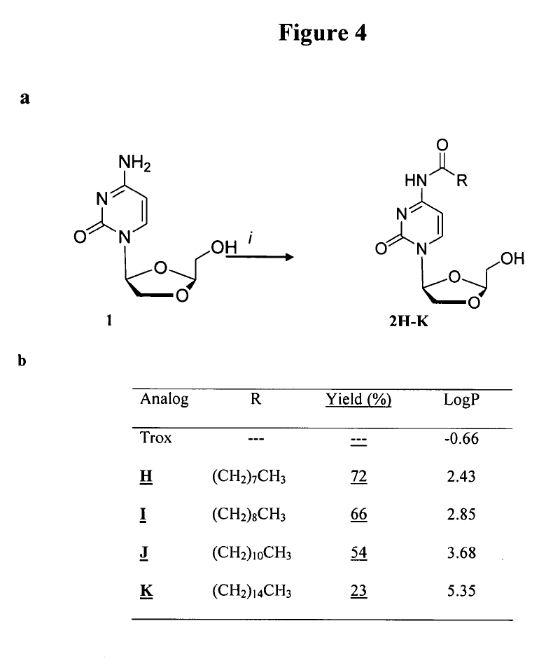

A small library of troxacitabine prodrugs was synthesised using a straightforward parallel solution-phase approach as described above. Troxacitabine, synthesized starting from L-gulose according to a well-known procedure^{[11]}, was dissolved in anhydrous methanol and treated, in an Argonaut Quest 210 organic synthesizer with different acid anhydrides. After 6 h at 55° C., the reaction mixtures were simply filtered, and then purified on a small silica flash column with gradient elution (hexanes:ethyl acetate), from which, troxacitabine prodrugs 2H-K were obtained in a white solid state. (See FIG. 4)

Sensitivity to four prodrugs with linear aliphatic chains of different length was determined by the SRB cytotoxicity assay^{[12]}, the IC₅₀ value of the drug was determined in the different cell lines by interpolating the growth inhibition curves. The tests were performed on the BxPC-3 and Panc-02 pancreatic cancer cell lines.

### Results and Discussion

Increasing the lipophilicity of troxacitabine by adding an aliphatic chain significantly enhances the sensitivity of pancreatic cancer cell lines to the drugs. The troxacitabine analogs I, J and K showed the greatest modulation compared to troxacitabine, in BxPC-3 analog J enhanced the sensitivity 160 fold and in Panc-02 analog I enhanced the sensitivity 1400 fold (FIG. 3). It seems that increasing the lipophilicity decreases the IC₅₀ to an optimum level at about (CH₂)₈, further increasing the lipophilicity does not seem to have a positive effect on the sensitivity in these cell lines. The effect of increased lipophilicity might be explained by increased influx further bypassing the nucleoside transporters or by an increased retention of the prodrugs in the cells. Prodrugs of Ara-C also containing an aliphatic side chain showed an increased activity in leukemic cell lines resistant to Ara-C. The aliphatic side chain length and to a lesser extent the amount of double bonds determined the activity of the compound, the compound with the shortest side chain (chain length: 16) and one double bond showed the best activity^{[13]}. Another lipophilic prodrug of Ara-c, NOAC, containing a C₁₈ aliphatic side chain, also showed increased activity in a xenograft model against leukemias and solid tumors^{[14]}. It has been shown that the speed of desorption from the membrane is related to the chain length of the fatty acid^{[15]}. This might explain why there seems to be an optimal lipophilicity after which no further enhancement of the drugs was observed. The exact mechanism of entry and retention in the cell of compounds containing aliphatic side chains may be further investigated.

### REFERENCES

### Set A for Non-Small Cell Lung Cancer

¹ Groove, C. L.; Guo, X.; Liu, S-H.; Gao, Z.; Chu, C. K.; Cheng, Y. C. Anticancer activity of a novel nucleoside analogue with the unnatural L configuration. Cancer Res. 1995, 55, 3008-3011.
² Kukhanova, M.; Liu, S-H.; Mozzherin, D.; Lin, T-S.; Chu, C. K.; Cheng, Y. C. L- and D-enantiomers of 2,3-dideoxycytidine 5-triphosphate analogs as substrates for human DNA polymerases. J. Biol. Chem. 1995, 270, 23055-23059.
³ Gourdeau, H.; Clarke, M. L.; Ouellet, F.; Mowles, D.; Selner, M.; Richard, A.; Lee, N.; Mackey, J. R.; Young, J. D.; Jolivet, J.; Lafreniere, R. G.; Cass, C. E. Mechanisms of uptake and resistance to troxacitabine, a novel deoxycytidine nucleoside analogue, in human leukemic and solid tumor cell lines. Cancer Res. 2001, 61, 7217-7224.
⁴ Gourdeau, H.; Bibeau, L.; Ouellet, F.; et al. Comparative study of a novel nucleoside analogue (Troxatyl™, troxacitabine, BCH-4556) and araC against leukemic human tumor xenografts expressing high or low cytidine deaminase activity. Cancer Chemoter. Pharmacol. 2001, 47, 236-240.
⁵ Giles, F. J.; Cortes, J. E.; Baker, S. D.; et al. Troxacitabine, a novel dioxolane nucleoside analog, has activity in patients with advanced leukaemia. J. Clin. Oncol. 2001, 19, 762-771.
⁶ De Bono, J. S.; Stephenson, J. Jr.; Baker, S. D.; Hidalgo, M.; Patnaik, A.; Hammond, L. A.; Weiss. G.; Goetz, A.; Siu, L.; Simmons, C.; Jolivet, J.; Rowinsky, E. K. Troxacitabine, an L-stereoisomeric nucleoside analog, on a five-times-daily schedule: a phase I and pharmacokinetic study in patients with advanced solid malignancies. J. Clin. Oncol. 2002, 1, 96-109.
⁷ Ettmayer, O.; Amidon, G. L.; Clement, B.; Testa, B. Lesson learned from marketed and investigational prodrugs. J. Med. Chem. 2004, 47, 2393-2404.
⁸ Walko C M et al, Clin ther, 2005, 27, 23-44
⁹ (a) Breistol K et al, cancer res, 1999, 59, 2944-9; (b) Bergman A M et al, biochem pharmacol, 2004, 67, 503-11.
¹⁰ Song, X.; Lorenzi, P. L.; Landowski, C. P.; Vig, B. S.; Hilfinger, J. M.; Amidon, G. L. Amino acid ester prodrugs of the anticancer agent gemcitabine: synthesis, bioconversion, metabolic bioevasion, and hPEPT1-mediated transport. Mol. Pharm. 2005, 2, 157-167.
¹¹ Tobias, S.; Borch, R. F. Synthesis and biological evaluation of cytarabine phosphoramidite prodrug. Mol. Pharm. 2003, 1, 122-116.
¹² a) Gorden, E. M.; Barrett, R. W.; Dower, W. S.; Foder, S. P. A.; Gallop, M. A. Applications of Combinatorial Technologies to Drug Discovery. 2. Combinatorial Organic Synthesis, Library Screening Strategies, and Future Directions. J. Med. Chem. 1994, 37, 1385-1401.
¹³ Thompson, L. A.; Ellman, J. A. Synthesis and Applications of Small Molecule Libraries. Chem. Rev. 1996, 96, 555-600.
¹⁴ Dolle, R. E. Comprehensive Survey of Combinatorial Library Synthesis: 2004. J. Comb. Chem. 2005, 7, 739-798.
¹⁵ Storer, R. Solution-phase synthesis in combinatorial chemistry: Applications in drug discovery. Drug Discovery Today 1996. 1,248-254.
¹⁶ Kim, H. O.; Schinazi, R. F.; Nampalli, S., Shanmuganathan, K.; Cannon, D. L.; Alves, A. J.; Jeong, L. S.; Beach, J. W.; Chu, C. K. 1,3-Dioxolanylpurine nucleosides (2S,4S) and (2S,4S) with selective anti-HIV-1 activity in human lymphocytes. J. Med. Chem. 1993, 36, 30-37.
¹⁷ Clarke, P. A.; Kayaleh, N. E.; Smith, M. A.; Baker, J. R.; Bird, S. J.; Chan, C. A one-step procedure for the monoacylation of symmetrical 1,2-diols. J. Org. Chem. 2002, 67, 5226-5231.
¹⁸ Kim, J; Jang, D. O. A convenient method for synthesis of symmetrical acid anhydrides from carboxylic acids with trichloroacetonitrile and triphenylphosphine. Synth. Commun. 2001, 31, 395-399.
¹⁹ Bergman A M et al, Br j canc, 2003, 88, 1963-70.
²⁰ Sigmond J, unpublished dataCX

### Set B for Pancreatic Cancer

1. Groove, K. L., Guo, X., Liu, S. H., Gao, Z., Chu, C. K., and Cheng, Y. C. 1995. Anticancer activity of beta-L-dioxolane-cytidine, a novel nucleoside analogue with the unnatural L configuration. Cancer Res., 55, 3008-3011.
2. Gourdeau, H. and Jolivet, J., 2006. Troxacitabine (Troxatyl™) A Deoxycytidine Nucleoside Analog With Potent Antitumor Activity in Cancer Drug Discovery and Developement: Deoxynucleoside Analogs in Cancer Therapy, ed. Peters, G. J., pp. 199-214, Humana Press Inc., Totowa; N.J.
3. Gumina, G., Chong, Y., and Chu, C. K., 2006. L-Nucleosides as Chemotherapeutic Agents in Cancer Drug Discovery and Developement: Deoxynucleoside Analogs in Cancer Therapy, ed. Peters, G. J., pp. 173-198, Humana Press Inc., Totowa, N.J.
4. Gourdeau, H., Clarke, M. L., Ouellet, F., Mowles, D., Selner, M., Richard, A., Lee, N., Mackey, I. R., Young, J. D., Jolivet, J., Lafreniere, R. G., and Cass, C. E. 2001. Mechanisms of uptake and resistance to troxacitabine, a novel deoxycytidine nucleoside analogue, in human leukemic and solid tumor cell lines. Cancer Res., 61, 7217-7224.
5. Kukhanova, M., Liu, S. H., Mozzherin, D., Lin, T. S., Chu, C. K., and Cheng, Y. C. 1995. L- and D-enantiomers of 2′,3′-dideoxycytidine 5′-triphosphate analogs as substrates for human DNA polymerases. Implications for the mechanism of toxicity. J. Biol. Chem., 270, 23055-23059.
6. Chou, K. M., Kukhanova, M., and Cheng, Y. C. 2000. A novel action of human apurinic/apyrimidinic endonuclease: excision of L-configuration deoxyribonucleoside analogs from the 3′ termini of DNA. J. Biol. Chem., 275, 31009-31015.
7. Chou, K. M. and Cheng, Y. C. 2003. The exonuclease activity of human apurinic/apyrimidinic endonuclease (APE1). Biochemical properties and inhibition by the natural dinucleotide Gp4G. J. Biol. Chem., 278, 18289-18296.
8. Weitman, S., Marty, J., Jolivet, J., Locas, C., and Von Hoff, D. D. 2000. The new dioxolane, (−)-2′-deoxy-3′-oxacytidine (BCH-4556, troxacitabine), has activity against pancreatic human tumor xenografts. Clin. Cancer Res., 6, 1574-1578.
9. Lapointe, R., Letoumeau, R., Steward, W., Hawkins, R. E., Batist, G., Vincent, M., Whittom, R., Eatock, M., Jolivet, J., and Moore, M. 2005. Phase II study of troxacitabihe in chemotherapy-naive patients with advanced cancer of the pancreas: gastrointestinal tumors. Ann. Oncol., 16, 289-293.
10. de Bono, J. S., Stephenson, J., Jr., Baker, S. D., Hidalgo, M., Patnaik, A., Hammond, L. A., Weiss, G., Goetz, A., Siu, L., Simmons, C., Jolivet, J., and Rowinsky, E. K. 2002. Troxacitabine, an L-stereoisomeric nucleoside analog, on a five-times-daily schedule: a phase I and pharmacokinetic study in patients with advanced solid malignancies. J. Clin. Oncol., 20, 96-109.
11. Kim, H. O., Schinazi, R. F., Nampalli, S., Shanmuganathan, K., Cannon, D. L., Alves, A. J., Jeong, L. S., Beach, J. W., and Chu, C. K. 1993. 1,3-dioxolanylpurine nucleosides (2R,4R) and (2R,4S) with selective anti-HIV-1 activity in human lymphocytes. J. Med. Chem., 36, 30-37.
12. Keepers, Y. P., Pizao, P. E., Peters, G. J., Ark-Otte, J., Winograd, B., and Pinedo, H. M. 1991. Comparison of the sulforhodamine B protein and tetrazolium (MTT) assays for in vitro chemosensitivity testing. Eur. J. Cancer, 27, 897-900.
13. Bergman, A. M., Kuiper, C. M., Voorn, D. A., Comijn, E. M., Myhren, F., Sandvold, M. L., Hendriks, H. R., and Peters, G. J. 2004. Antiproliferative activity and mechanism of action of fatty acid derivatives of arabinofuranosylcytosine in leukemia and solid tumor cell lines. Biochem. Pharmacol., 67, 503-511.
14. Koller-Lucae, S. K., Suter, M. J., Rentsch, K. M., Schott, H., and Schwendener, R. A. 1999. Metabolism of the new liposomal anticancer drug N4-octadecyl-1-beta-D-arabinofuranosylcytosine in mice. Drug Metab Dispos., 27, 342-350.
15. Hamilton, J. A. 2003. Fast flip-flop of cholesterol and fatty acids in membranes: implications for membrane transport proteins. Curr. Opin. Lipidol., 14, 263-271.

1. A method of treating cancer in a patient in need thereof comprising administering an effective amount of at least one compound according to the chemical structure: 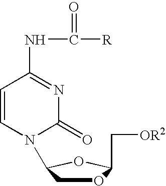
Wherein R is an optionally substituted C₃-C₇ cyclic hydrocarbon, an optionally substituted C₁-C₂₂ straight or branch-chained alkyl group, or an optionally substituted phenyl group;
R² is H or a mono-, di- or triphosphosphate group as a fully or partially protonated species, or a phosphodiester group, or a pharmaceutically acceptable salt thereof, optionally in combination with a pharmaceutically acceptable carrier, additive or excipient.

2. The method according to claim 1 wherein said cancer is a solid tumor.

3. The method according to claim 1 wherein said cancer is non-small cell lung cancer.

4. The method according to claim 1 wherein said cancer is pancreatic cancer.

5. The method according to claim 1 wherein said cancer is a cancer of the stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma.

6. The method according to claim 1 wherein said compound is coadministered to said patient along with an effective amount of a compound selected from the group consisting of Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; bortezomib; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubiCin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; ixabepilone; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; photophrin, pipobroman; plicamycin; mithramycin; porfimer sodium; procarbazine; quinacrine; Rasburicase; Rituximab; Sargramostim; streptozocin; talbuvidine (LDT); talc; tamoxifen; tarceva; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof.

7. The method according to claim 5 wherein said compound is coadministered to said patient along with an effective amount of a compound selected from the group consisting of Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; bortezomib; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; ixabepilone; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; photophrin, pipobroman; plicamycin; mithramycin; porfimer sodium; procarbazine; quinacrine; Rasburicase; Rituximab; Sargramostim; streptozocin; talbuvidine (LDT); talc; tamoxifen; tarceva; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof.

8. A method of treating non-small cell lung cancer in a patient in need thereof comprising administering to a patient in need of therapy an effective amount of at least one compound according to the chemical structure: 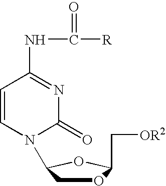
Wherein R is an optionally substituted C₃-C₇ cyclic hydrocarbon, an optionally substituted C₁-C₂₂ straight or branch-chained alkyl group, or an optionally substituted phenyl group;
R² is H or a mono-, di- or triphosphosphate group as a fully or partially protonated species, or a phosphodiester group, or a pharmaceutically acceptable salt thereof, optionally in combination with a pharmaceutically acceptable carrier, additive or excipient.

9. The method according to claim 8 wherein R is a C₃-C₆ cycloalkyl group.

10. The method according to claim 8 wherein R is a C₁-C₁₅ linear or branched-chain alkyl group.

11. The method according to claim 8 wherein R is an optionally substituted phenyl group wherein said phenyl group, when substituted, is substituted with one or two groups selected from the group consisting of F, Cl, Br, OMe or mixtures thereof.

12. The method according to claim 8 wherein R² is H or a phosphate group in the free acid or salt form.

13. The method according to claim 9 wherein R² is H or a phosphate group in the free acid or salt form.

14. The method according to claim 10 wherein R² is H or a phosphate group in the free acid or salt form.

15. The method according to claim 11 wherein R² is H or a phosphate group in the free acid or salt form.

16. The method according to claim 8 wherein said compound is coadministered to said patient with a compound(s) selected from the group consisting of ixabepilone, bortezomib, alone or in combination with docetaxel, photofrin, taxol, alone or in combination with cisplatin, gemcitabine, tarceva, and mixtures thereof.

17. A method of treating pancreatic cancer in a patient in need thereof comprising administering to a patient in need of therapy an effective amount of at least one compound according to the chemical structure: 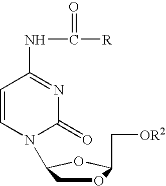
Wherein R is an optionally substituted C₃-C₇ cyclic hydrocarbon, an optionally substituted C₁-C₂₂ straight or branch-chained alkyl group, or an optionally substituted phenyl group;
R² is H or a mono-, di- or triphosphosphate group as a fully or partially protonated species, or a phosphodiester group, or a pharmaceutically acceptable salt thereof, optionally in combination with a pharmaceutically acceptable carrier, additive or excipient.

18. The method according to claim 17 wherein R is a C₈ to C₁₅ linear alkyl group.

19. The method according to claim 17 wherein R is an optionally substituted phenyl group.

20. The method according to claim 17 wherein R² is H or a phosphate group in the free acid or salt form.

21. The method according to claim 18 wherein R² is H or a phosphate group in the free acid or salt form.

22. The method according to claim 17 wherein said compound is coadministered to said patient with an additional compound(s) selected from the group consisting of tarceva, alone or in combination with gemcitabine, apigenin, MGN-3, EGCG, or an analgesic agent.

23-37. (canceled)

38. A combination pharmaceutical composition comprising an effective amount of at least one compound according to the chemical structure: 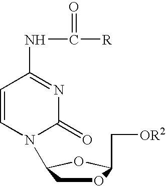
Wherein R is an optionally substituted C₃-C₇ cyclic hydrocarbon, an optionally substituted C₁-C₂₂ straight or branch-chained alkyl group, or an optionally substituted phenyl group;
R² is H or a mono-, di- or triphosphosphate group as a fully or partially protonated species, or a phosphodiester group, or a pharmaceutically acceptable salt thereof, a pharmaceutically acceptable carrier, additive or excipient and at least one additional compound selected from the group consisting of Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; bortezomib; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; ixabepilone; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; photophrin, pipobroman; plicamycin; mithramycin; porfimer sodium; procarbazine; quinacrine; Rasburicase; Rituximab; Sargramostim; streptozocin; talbuvidine (LDT); talc; tamoxifen; tarceva; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof.

39. The composition according to claim 38 wherein said additional compound is selected from the group consisting of ixabepilone, bortezomib, docetaxel, photofrin, taxol, cisplatin, gemcitabine, tarceva and mixtures thereof.
